(19) United States
(12) Patent Application Publication
(10) Pub. No.: US 2007/0140975 A1
(43) Pub. Date: Jun. 21, 2007

(54) Opioid formulations having reduced potential for abuse

(75) Inventors: Anand R. Baichwal, Wappingers Falls, NY (US); Paul M. Woodcock, Brookfield, CT (US); Troy McCall, Germantown, TN (US)

Correspondence Address: WILMER CUTLER PICKERING HALE AND DORR LLP, 60 STATE STREET, BOSTON, MA, 02109, US

(73) Assignee: Penwest Pharmaceuticals Co., Danbury, CT (US)

(21) Appl. No.: 11/704,848

(22) Filed: Feb. 9, 2007

Related U.S. Application Data
(63) Continuation of application No. 10/254,207, filed on Sep. 25, 2002.
(60) Provisional application No. 60/324,546, filed on Sep. 26, 2001.

(51) Int. Cl. A61K 9/44; A61K 9/26

(52) U.S. Cl. 424/10.2; 424/469

(57) ABSTRACT

The invention provides opioid formulations having reduced potential for abuse, and having reduced potential for illegal sale and distribution. The opioid formulations of the invention comprise at least one opioid and a sustained release delivery system.

### REFERENCE TO RELATED APPLICATIONS

This application is a continuation of U.S. application Ser. No. 10/254,207, filed Sep. 25, 2002, which claims the benefit of U.S. Provisional Application No. 60/324,546, filed Sep. 26, 2001. The above-identified applications are incorporated herein by reference in their entireties.

### FIELD OF THE INVENTION

The invention provides opioid formulations having reduced potential for abuse, and having reduced potential for illegal sale and distribution. The opioid formulations of the invention comprise at least one opioid and a sustained release delivery system.

### BACKGROUND OF THE INVENTION

One concern associated with the use of some pharmaceuticals, such as opioids (e.g., OxyContin®), is the unprescribed abuse of the drugs by the patient or the diversion of the drugs from the patient to another person for recreational purposes, e.g., to an addict. A number of factors govern abuse of pharmaceuticals, such as opioids, including the capacity of the drug to produce the kind of physical dependence in which drug withdrawal causes sufficient distress to bring about drug-seeking behavior; the ability to suppress withdrawal symptoms caused by withdrawal from other agents; the degree to which it induces euphoria (e.g., similar to that produced by morphine and other opioids); the patterns of toxicity that occur when the drug is dosed above its normal therapeutic range; and physical characteristics of the drugs, such as water solubility. The physical characteristics of the drug may determine whether the drug is likely to be abused by inhalation or parenteral routes.

Extended release versions of pharmaceutical formulations, such as opioids, often incorporate higher levels of the active material than are found in immediate release versions of the same product and are therefore particularly attractive to drug addicts or recreational drug users. The higher levels of drug can be made available by crushing or grinding the tablet into a fine powder that destroys the complex delivery system afforded by the intact tablet. The powder can then be inhaled through the oral-pharyngeal tract or snorted through the nasal-pharyngeal tract. Alternatively, the powder can be reconstituted in a small volume of water and injected into the body using a hypodermic needle.

There is a need in the art for pharmaceutical formulations that have reduced potential for abuse when compared to currently available formulations. The invention is directed to this, as well as other, important ends.

### SUMMARY OF THE INVENTION

The invention provides methods for reducing the potential for drug abuse by prescribing and/or administering to patients an effective amount of an abuse-potential drug formulation or kits of the invention to treat pain. The abuse-potential drug formulations and kits of the invention have significantly less potential for abuse when compared to commercially available formulations. An abuse-potential drug comprises an opioid compound.

The invention also provides methods for reducing the illegal sale and/or distribution of drugs by prescribing and/or administering to patients an effective amount of the abuse-potential drug formulations or kits of the invention to treat pain. The abuse-potential drug formulations and kits of the invention have significantly less potential for illegal sale and/or distribution when compared to commercially available formulations because of their significantly reduced potential for abuse. An abuse-potential drug comprises an opioid compound.

These and other aspects of the invention are described in detail herein.

### DETAILED DESCRIPTION OF THE INVENTION

The invention provides compositions comprising at least one abuse-potential drug and a sustained release delivery system, where the sustained release delivery system comprises (i) at least one hydrophilic compound, at least one cross-linking agent, and at least one pharmaceutical diluent; (ii) at least one hydrophilic compound, at least one cross-linking agent, at least one pharmaceutical diluent, and at least one hydrophobic polymer; (iii) at least one hydrophilic compound, at least one cross-linking agent, at least one pharmaceutical diluent, and at least one cationic cross-linking agent; (iv) at least one hydrophilic compound, at least one cross-linking agent, at least one pharmaceutical diluent, at least one cationic cross-linking compound, and at least one hydrophobic polymer; (v) at least one hydrophilic compound, at least one cationic cross-linking compound, and at least one pharmaceutical diluent; or (vi) at least one hydrophilic compound, at least one cationic cross-linking compound, at least one pharmaceutical diluent, and at least one hydrophobic compound.

In one aspect of the invention, the invention comprises at least one opioid and a sustained release delivery system, where the sustained release delivery system comprises (i) at least one hydrophilic compound, at least one cross-linking agent, and at least one pharmaceutical diluent; (ii) at least one hydrophilic compound, at least one cross-linking agent, at least one pharmaceutical diluent, and at least one hydrophobic polymer; (iii) at least one hydrophilic compound, at least one cross-linking agent, at least one pharmaceutical diluent, and at least one cationic cross-linking agent; (iv) at least one hydrophilic compound, at least one cross-linking agent, at least one pharmaceutical diluent, at least one cationic cross-linking compound, and at least one hydrophobic polymer; (v) at least one hydrophilic compound, at least one cationic cross-linking compound, and at least one pharmaceutical diluent; or (vi) at least one hydrophilic compound, at least one cationic cross-linking compound, at least one pharmaceutical diluent, and at least one hydrophobic compound.

In another aspect, the invention provides compositions comprising at least one abuse-potential drug and a sustained release delivery system. The abuse-potential drug may be homogeneously dispersed in the sustained release delivery system. The abuse-potential drug may be present in the composition in an amount of about 0.5 milligrams to about 1000 milligrams, preferably in an amount of about 1 milligram to about 800 milligrams, still more preferably in an amount of about 1 milligram to about 200 milligrams, most preferably in an amount of about 1 milligram to about 100 milligrams.

Another aspect of the invention provides compositions comprising at least one opioid and a sustained release delivery system. The opioid may be homogeneously dispersed in the sustained release delivery system. The opioid may be present in the composition in an amount of about 0.5 milligrams to about 1000 milligrams, preferably in an amount of about 1 milligram to about 800 milligrams, still more preferably in an amount of about 1 milligram to about 200 milligrams, most preferably in an amount of about 1 milligram to about 100 milligrams.

The term “abuse-potential drug” includes pharmaceutically active substances having the capacity to produce the kind of physical dependence in which drug withdrawal causes sufficient distress to bring about drug-seeking behavior; the ability to suppress withdrawal symptoms caused by withdrawal from other agents; the degree to which it induces euphoria (e.g., similar to that produced by morphine and other opioids); the patterns of toxicity that occur when the drug is dosed above its normal therapeutic range; and physical characteristics of the drugs, such as water solubility. The physical characteristics of the drug may determine whether the drug is likely to be abused by inhalation or parenteral routes. An abuse-potential drug includes stereoisomers thereof, metabolites thereof, salts thereof, ethers thereof, esters thereof and/or derivatives thereof (preferably pharmaceutically acceptable salts thereof). An opioid is a preferred embodiment of an abuse-potential drug. Other narcotics are apparent to those of ordinary skill in the art and are understood to fall within the scope of the invention.

The term “opioid” includes stereoisomers thereof, metabolites thereof, salts thereof, ethers thereof, esters thereof and/or derivatives thereof (preferably pharmaceutically acceptable salts thereof). The opioids may be mu-antagonists and/or mixed mu-agonists/antagonists. Exemplary opioids include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, stereoisomers thereof, metabolites thereof, salts thereof, ethers thereof, esters thereof, and/or derivatives thereof. In preferred embodiments, the opioid is morphine, codeine, hydromorphone, hydrocodone, oxycodone, dihydrocodeine, dihydromorphine, oxymorphone, 6-hydroxyoxymorphone (including 6-α-hydroxyoxymorphone and/or 6-β-hydroxyoxymorphone), or tramadol.

The abuse-potential drug or opioid may be in the form of any pharmaceutically acceptable salt known in the art. Exemplary pharmaceutically acceptable salts include hydrochloric, sulfuric, nitric, phosphoric, hydrobromic, maleric, malic, ascorbic, citric, tartaric, pamoic, lauric, stearic, palmitic, oleic, myristic, lauryl sulfuric, napthalinesulfonic, linoleic, linolenic acid, and the like.

The sustained release delivery system comprises at least one hydrophilic compound. The hydrophilic compound preferably forms a gel matrix that releases the opioid at a sustained rate upon exposure to liquids. As used herein, “liquids” includes, for example, gastrointestinal fluids, aqueous solutions (such as those used for in vitro dissolution testing), and mucosas (e.g., of the mouth, nose, lungs, esophagus, and the like). The rate of release of the opioid from the gel matrix depends on the drug's partition coefficient between the components of the gel matrix and the aqueous phase within the gastrointestinal tract. In the compositions of the invention, the weight ratio of opioid to hydrophilic compound is generally in the range of about 1:0.5 to about 1:25, preferably in the range of about 1:0.5 to about 1:20. The sustained release delivery system generally comprises the hydrophilic compound in an amount of about 20% to about 80% by weight, preferably in an amount of about 20% to about 60% by weight, more preferably in an amount of about 40% to about 60% by weight, still more preferably in an amount of about 50% by weight.

The hydrophilic compound may be any known in the art. Exemplary hydrophilic compounds include gums, cellulose ethers, acrylic resins, polyvinyl pyrrolidone, protein-derived compounds, and mixtures thereof. Exemplary gums include heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include hydroxyalkyl celluloses and carboxyalkyl celluloses. Preferred cellulose ethers include hydroxyethyl celluloses, hydroxypropyl celluloses, hydroxypropylmethyl-celluloses, carboxy methylcelluloses, and mixtures thereof Exemplary acrylic resins include polymers and copolymers of acrylic acid, methacrylic acid, methyl acrylate and methyl methacrylate. In some embodiments, the hydrophilic compound is preferably a gum, more preferably a heteropolysaccharide gum, most preferably a xanthan gum or derivative thereof. Derivatives of xanthan gum include, for example, deacylated xanthan gum, the carboxymethyl esters of xanthan gum, and the propylene glycol esters of xanthan gum.

In another embodiment, the sustained release delivery system may further comprise at least one cross-linking agent. The cross-linking agent is preferably a compound that is capable of cross-linking the hydrophilic compound to form a gel matrix in the presence of liquids. The sustained release delivery system generally comprises the cross-linking agent in an amount of about 0.5% to about 80% by weight, preferably in an amount of about 2% to about 54% by weight, more preferably in an amount of about 20% to about 30% by weight more, still more preferably in an amount of about 25% by weight.

Exemplary cross-linking agents include homopolysaccharides. Exemplary homopolysaccharides include galactomannan gums, such as guar gum, hydroxypropyl guar gum, and locust bean gum. In some embodiments, the cross-linking agent is preferably a locust bean gum or a guar gum. In other embodiments, the cross-linking agents may be alginic acid derivatives or hydrocolloids.

When the sustained release delivery system comprises at least one hydrophilic compound and at least one cross-linking agent, the ratio of hydrophilic compound to cross-linking agent may be from about 1:9 to about 9: 1, preferably from about 1:3 to about 3:1.

The sustained release delivery system of the invention may further comprise one or more cationic cross-linking compounds. The cationic cross-linking compound may be used instead of or in addition to the cross-linking agent. The cationic cross-linking compounds may be used in an amount sufficient to cross-link the hydrophilic compound to form a gel matrix in the presence of liquids. The cationic cross-linking compound is present in the sustained release delivery system in an amount of about 0.5% to about 30% by weight, preferably from about 5% to about 20% by weight.

Exemplary cationic cross-linking compounds include monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound may be one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system comprises at least one hydrophilic compound and at least one cationic cross-linking compound, the ratio of hydrophilic compound to cationic cross-linking compound may be from about 1:9 to about 9: 1, preferably from about 1:3 to about 3:1.

Two properties of compounds (e.g., the at least one hydrophilic compound and the at least one cross-linking agent; or the at least one hydrophilic compound and at least one cationic cross-linking compound) that form a gel matrix upon exposure to liquids are fast hydration of the compounds/agents and a gel matrix having a high gel strength. These two properties, which are needed to achieve a slow release gel matrix, are maximized in the invention by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby act synergistically to provide a higher than expected viscosity (i.e., high gel strength) of the gel matrix.

The sustained release delivery system may further comprise one or more pharmaceutical diluents known in the art. Exemplary pharmaceutical diluents include monosaccharides, disaccharides, polyhydric alcohols and mixtures thereof. Preferred pharmaceutical diluents include, for example, starch, lactose, dextrose, sucrose, microcrystalline cellulose, sorbitol, xylitol, fructose, and mixtures thereof. In other embodiments, the pharmaceutical diluent is water-soluble, such as lactose, dextrose, sucrose, or mixtures thereof. The ratio of pharmaceutical diluent to hydrophilic compound is generally from about 1:8 to about 8: 1, preferably from about 1:3 to about 3:1. The sustained release delivery system generally comprises one or more pharmaceutical diluents in an amount of about 20% to about 80% by weight, preferably about 35% by weight. In other embodiments, the sustained release delivery system comprises one or more pharmaceutical diluents in an amount of about 40% to about 80% by weight.

The sustained release delivery system of the invention may further comprise one or more hydrophobic polymers. The hydrophobic polymers may be used in an amount sufficient to slow the hydration of the hydrophilic compound without disrupting it. For example, the hydrophobic polymer may be present in the sustained release delivery system in an amount of about 0.5% to about 20% by weight, preferably in an amount of about 2% to about 10% by weight, more preferably in an amount of about 3% to about 7% by weight, still more preferably in an amount of about 5% by weight.

Exemplary hydrophobic polymers include alkyl celluloses (e.g., C₁₋₆ alkyl celluloses, carboxymethylcellulose), other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes, shellac, hydrogenated vegetable oils, and mixtures thereof. The hydrophobic polymer is preferably, methyl cellulose, ethyl cellulose or propyl cellulose, more preferably ethyl cellulose.

The compositions of the invention may be further admixed with one or more wetting agents (such as polyethoxylated castor oil, polyethoxylated hydrogenated castor oil, polyethoxylated fatty acid from castor oil, polyethoxylated fatty acid from hydrogenated castor oil) one or more lubricants (such as magnesium stearate), one or more buffering agents, one or more colorants, and/or other conventional ingredients.

The sustained release formulations comprising at least one opioid are preferably orally administrable solid dosage formulations which may be, for example, tablets, capsules comprising a plurality of granules, sublingual tablets, powders, or granules; preferably tablets. The tablets may have an enteric coating or a hydrophilic coating.

The sustained release delivery system in the compositions of the invention may be prepared by dry granulation or wet granulation, before the opioid is added, although the components may be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the opioid and, optionally, one or more wetting agents, one or more lubricants, one or more buffering agents, one or more coloring agents, or other conventional ingredients, to produce a granulated composition. The sustained release delivery system and the opioid may be blended with, for example, a high shear mixer. The opioid is preferably finely and homogeneously dispersed in the sustained release delivery system. The granulated composition, in an amount sufficient to make a uniform batch of tablets, is subjected to tableting in a conventional production scale tableting machine at normal compression pressures, i.e., about 2,000-16,000 psi. The mixture should not be compressed to a point where there is subsequent difficulty with hydration upon exposure to liquids. Methods for preparing sustained release delivery systems are described in U.S. Pat. Nos. 4,994,276, 5,128,143, 5,135,757, 5,455,046, 5,512,297 and 5,554,387, the disclosures of which are incorporated by reference herein in their entirety.

The average particle size of the granulated composition is from about 50 microns to about 400 microns, preferably from about 185 microns to about 265 microns. The average density of the granulated composition is from about 0.3 g/ml to about 0.8 g/ml, preferably from about 0.5 g/ml to about 0.7 g/ml. The tablets formed from the granulations are generally from about 6 to about 8 kg hardness. The average flow of the granulations are from about 25 to about 40 g/sec.

In other embodiments, the invention provides sustained release coatings over an inner core comprising at least one opioid. For example, the inner core comprising the opioid may be coated with a sustained release film, which, upon exposure to liquids, releases the opioid from the core at a sustained rate.

In one embodiment, the sustained release coating comprises at least one water insoluble compound. The water insoluble compound is preferably a hydrophobic polymer. The hydrophobic polymer may be the same as or different from the hydrophobic polymer used in the sustained release delivery system. Exemplary hydrophobic polymers include alkyl celluloses (e.g., C₁₋₆ alkyl celluloses, carboxymethylcellulose), other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes (alone or in admixture with fatty alcohols), shellac, hydrogenated vegetable oils, and mixtures thereof. The hydrophobic polymer is preferably, methyl cellulose, ethyl cellulose or propyl cellulose, more preferably ethyl cellulose. The sustained release formulations of the invention may be coated with a water insoluble compound to a weight gain from about 1 to about 20% by weight.

The sustained release coating may further comprise at least one plasticizer such as triethyl citrate, dibutyl phthalate, propylene glycol, polyethylene glycol, or mixtures thereof.

The sustained release coating may also contain at least one water soluble compound, such as polyvinylpyrrolidones, hydroxypropylmethylcelluloses, or mixtures thereof. The sustained release coating may comprise at least one water soluble compound in an amount from about 1% to about 6% by weight, preferably in an amount of about 3% by weight.

The sustained release coating may be applied to the opioid core by spraying an aqueous dispersion of the water insoluble compound onto the opioid core. The opioid core may be a granulated composition made, for example, by dry or wet granulation of mixed powders of opioid and at least one binding agent; by coating an inert bead with an opioid and at least one binding agent; or by spheronizing mixed powders of an opioid and at least one spheronizing agent. Exemplary binding agents include hydroxypropylmethylcelluloses. Exemplary spheronizing agents include microcrystalline celluloses. The inner core may be a tablet made by compressing the granules or by compressing a powder comprising an opioid.

In other embodiments, the compositions comprising at least one opioid and a sustained release delivery system, as described herein, are coated with a sustained release coating, as described herein. In still other embodiments, the compositions comprising at least one opioid and a sustained release delivery system, as described herein, are coated with a hydrophobic polymer, as described herein. In still other embodiments, the compositions comprising at least one opioid and a sustained release delivery system, as described herein, are coated with an enteric coating, such as cellulose acetate phthalate, hydroxypropylmethylcellulose phthalate, polyvinylacetate phthalate, methacrylic acid copolymer, shellac, hydroxypropylmethylcellulose succinate, cellulose acetate trimelliate, or mixtures thereof. In still other embodiments, the compositions comprising at least one opioid and a sustained release delivery system, as described herein, are coated with a hydrophobic polymer, as described herein, and further coated with an enteric coating, as described herein. In any of the embodiments described herein, the compositions comprising the opioid and a sustained release delivery system, as described herein, may optionally be coated with a hydrophilic coating which may be applied above or beneath the sustained release film, above or beneath the hydrophobic coating, and/or above or beneath the enteric coating. Preferred hydrophilic coatings comprise hydroxypropylmethylcellulose.

Without intending to be bound by any theory of the invention, upon oral ingestion of the opioid sustained release formulation and contact of the formulation with gastrointestinal fluids, the sustained release formulation swells and gels to form a hydrophilic gel matrix from which the opioid is released. The swelling of the gel matrix causes a reduction in the bulk density of the formulation and provides the buoyancy necessary to allow the gel matrix to float on the stomach contents to provide a slow delivery of the opioid. The hydrophilic matrix, the size of which is dependent upon the size of the original formulation, can swell considerably and become obstructed near the opening of the pylorus. Since the opioid is dispersed throughout the formulation (and consequently throughout the gel matrix), a constant amount of opioid can be released per unit time in vivo by dispersion or erosion of the outer portions of the hydrophilic gel matrix. This phenomenon is referred to as a zero order release profile or zero order kinetics. The process continues, with the gel matrix remaining buoyant in the stomach, until substantially all of the opioid is released.

Without intending to be bound by any theory of the invention, the chemistry of certain of the components of the formulation, such as the hydrophilic compound (e.g., xanthan gum), is such that the components are considered to be self-buffering agents which are substantially insensitive to the solubility of the opioids and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, it may be possible that the sustained release formulation could potentially loosely interact with the mucin in the gastrointestinal tract and thereby provide another mode by which a constant rate of delivery of the opioid is achieved.

The two phenomenon discussed above (hydrophilic gel matrix and muco-adhesive properties) are possible mechanisms by which the sustained release formulations of the invention could interact with the mucin and fluids of the gastrointestinal tract and provide a constant rate of delivery of the opioids.

It has now been unexpectedly discovered that the two phenomenon discussed above (hydrophilic gel matrix and muco-adhesive properties) could be relied upon to produce formulations that will reduce or eliminate the abuse of opioids. In particular, the opioid formulations of the invention have significantly less potential for abuse than conventional opioid formulations.

If the opioid formulation of the invention is chewed or ground up for oral ingestion/inhalation (e.g., an oral-pharynx route), the formulation will swell and form a hydrophilic gel matrix that has muco-adhesive properties upon contact with the moist lining of the mucosa in the mouth and/or esophagus. The time available for absorption of drugs via the oral route is limited due to the rapid clearance of the surface coating of the mucosa in the mouth and esophagus. Therefore, if a patient attempts to abuse the opioid formulation of the invention by oral ingestion/inhalation, the opioid formulation of the invention will not reside in the mouth and/or esophagus long enough for absorption to take place. Moreover, the opioid, which is homogeneously distributed throughout the formulation of the invention, will substantially maintain its sustained release properties and will slowly release from the resulting hydrophilic gel matrix. Due to the slow release and muco-adhesive properties of the opioid formulations of the invention, the patient (e.g., drug addict) would not experience the euphoria that would be immediately available by abusing conventional opioid formulations by oral inhalation/ingestion. Accordingly, the opioid formulations of the invention would not be abused by patients or their potential for abuse would be significantly reduced (e.g., when compared to conventional opioid formulations).

If the opioid formulation of the invention is ground up for nasal inhalation (e.g., a nasal-pharynx route), the formulation will swell and form a hydrophilic gel matrix that has muco-adhesive properties upon contact with the moist lining of the mucosa in the nose, esophagus, and/or lungs. The time available for absorption of drugs via the nasal route is limited due to the rapid clearance of the surface coating of the mucosa in the nose. Therefore, if a patient attempts to abuse the opioid formulation of the invention by nasal inhalation, the opioid formulation of the invention will not reside in the nose long enough for absorption to take place. Moreover, the opioid, which is homogeneously distributed throughout the formulation of the invention, will maintain its sustained release properties and will slowly release from the resulting hydrophilic gel matrix. Due to the slow release and muco-adhesive properties of the opioid formulations of the invention, the patient (e.g., drug addict) would not experience the euphoria that would be immediately available by abusing conventional opioid formulations by nasal inhalation. Accordingly, the opioid formulations of the invention would not be abused or their potential for abuse would be significantly reduced (e.g., when compared to conventional opioid formulations).

If the opioid formulation of the invention is ground up to be administered parenterally (e.g., subcutaneous injection, intravenous injection, intra-arterial injection, intramuscular injection, intrastemal injection, infusion techniques), the formulation will swell and form a hydrophilic gel matrix that has muco-adhesive properties upon contact with water or other liquids. The high viscosity of the resulting hydrophilic gel matrix significantly reduces the ability for the material to be drawn into a syringe and/or forced through a syringe and into the skin for parenteral administration. Accordingly, the opioid formulations of the invention would not be abused or their potential for abuse would be significantly reduced (e.g., when compared to conventional opioid formulations).

Moreover, even if the opioid formulations of the invention were administered parenterally, the opioid, which is homogeneously distributed throughout the formulation, will maintain its sustained release properties and will slowly release from the resulting hydrophilic gel matrix. The patient (e.g., drug addict) would not experience the euphoria that would be immediately available by abusing conventional opioid formulations by parenteral administration. Accordingly, the opioid formulations of the invention would not be abused or their potential for abuse would be significantly reduced (e.g., when compared to conventional opioid formulations).

In view of the decreased potential for abuse of the opioid formulations of the invention for the reasons discussed above, the opioid formulations of the invention will less likely be illegally distributed and/or sold because they do not provide the euphoria that drug addicts or recreational drug users are seeking.

The invention provides methods for treating pain by prescribing and/or administering an effective amount of the sustained release formulations of opioids to a patient in need thereof. An effective amount is an amount sufficient to eliminate all pain or to alleviate the pain (i.e., reduce the pain compared to the pain present prior to administration of the opioid sustained release formulation).

“Sustained release” means that the opioid is released from the formulation at a controlled rate so that therapeutically beneficial blood levels (but below toxic levels) of the opioid are maintained over an extended period of time. The sustained release formulations of opioids are administered in an amount sufficient to alleviate pain for an extended period of time, preferably about 8 hours to about 24 hours, more preferably for a period of about 12 hours to about 24 hours. The opioid sustained release oral solid dosage formulations of the invention may be administered one to four times a day, preferably once or twice daily, more preferably once daily.

The pain may be minor to moderate to severe, and is preferably moderate to severe. The pain may be acute or chronic. The pain may be associated with, for example, cancer, autoimmune diseases, infections, surgical traumas, or accidental traumas. The patient may be an animal, preferably a mammal, more preferably a human.

While the compositions of the invention may be administered as the sole active pharmaceutical composition in the methods described herein, they can also be used in combination with one or more compounds/compositions that are known to be therapeutically effective against pain.

The invention provides pharmaceutical kits comprising one or more of the abuse-potential drug formulations of the invention. The invention provides pharmaceutical kits comprising one or more containers filled with one or more of the opioid formulations of the invention. The kits may further comprise other pharmaceutical compounds known in the art to be therapeutically effective against pain, and instructions for use. The kits of the invention reduce the potential of opioid abuse because they comprise the opioid formulations of the invention. The kits of the invention also reduce the potential for illegal sales and/or distribution of opioids because they contain the opioid formulations of the invention that have significantly reduced potential for abuse when compared to conventional opioid formulations. Because the kits of the invention have significantly reduced potential for illegal sales and/or distribution, the kits of the invention are also less likely to be stolen from manufacturers, pharmacies and doctors'offices by drug addicts who resort to theft to support their addictions.

### BRIEF DESCRIPTION OF THE DRAWINGS

FIG. 1 is a graphic depiction of the dissolution profiles of Formulation 1, Formulation 2, and Formulation 3.

### EXAMPLES

The following examples are for purposes of illustration only and are not intended to limit the scope of the appended claims.

A sustained release formulation of the invention was prepared by first screening Albuterol Sulfate, Lactose, and Syloid 244 separately through a #30 Mesh sieve (hereinafter “Formulation 1”). Albuterol Sulfate and TIMERx N® ((Penwest Pharmaceuticals Co., Patterson, N.Y.) were blended for ten minutes in a Patterson-Kelley P/K Blendmaster V-Blender. Lactose, Syloid 244 (synthetic amorphous silica, Grace Davison, Columbia, Md.) and Pruv™ (Sodium Stearyl Fumarate, NF, Penwest Pharmaceuticals Co., Patterson, N.Y.) were added to this mixture successively, blending for five minutes between each addition. The blended granulation was compressed to 217.0 mg and 10 Kp hardness on a tablet press using a Stokes RB-2 5/16″ round standard concave beveled edge. The final tablet composition is listed below:

| Component | % | mg/tab |
|---|---|---|
| Albuterol Sulfate | 3.4 | 9.6 |
| TIMERx N | 71.1 | 160.0 |
| Lactose | 17.8 | 40.1 |
| Syloid 244 | 1.9 | 4.3 |
| Pruv | 1.9 | 3.0 |

A second formulation with release modifying properties was prepared as a control using Eudragit® RL30D (Röhm, Malden, Mass.) (hereinafter “Formulation 2”). Eudragit® RL30D is an aqueous dispersion of copolymers of acrylic and methacrylic acid esters with a low content of quaternary ammonium groups with a mean molecular weight of approximately 150,000. Albuterol Sulfate and Lactose were dispensed into a Niro Aeromatic Strea-1 Fluid Bed Dryer and the material was preheated and fluidized. During fluidization, Eudragit RL30D was added by spraying. This composition was allowed to dry in the fluid bed dryer until the Loss on Drying (LOD) was less than one percent. The dried granulation was screened though a #16 Mesh sieve, then placed in an Aeromatic Fielder PP-1 High Shear Granulator equipped with a 10 L bowl. Meanwhile, Stearyl Alcohol was melted. While running the impeller at low speed, the melted Stearyl Alcohol was added; mixing was continued to achieve uniform distribution.

Granulation continued at high speed until proper granules were formed, then the granules were cooled to room temperature. The cooled granules were screened through a #16 Mesh sieve and dispensed into a Patterson-Kelley P/K Blendmaster V-Blender. Stearic acid was added and the mixture was blended for five minutes. Talc was added and the mixture was blended for an additional five minutes. The blended granulation was compressed to 281.4 mg and 10 Kp hardness on a tablet press using a Stokes RB-2 5/16″ round standard concave beveled edge. The final tablet composition is listed below:

| Component | % | mg/tab |
|---|---|---|
| Albuterol Sulfate | 3.4 | 9.6 |
| Lactose | 71.1 | 200.0 |
| Stearyl Alcohol | 17.8 | 61.2 |
| Stearic Acid | 1.9 | 5.3 |
| Talc | 1.9 | 5.3 |
| Eudragit RL30D | 4.0 | 11.2 |

A third formulation was prepared in water as a control (hereinafter “Formulation 3”). Albuterol Sulfate and Lactose were mixed in a bowl mixer for one minute. While running the impeller at low speed, water was added to the mixture over a one minute interval. The mixture was granulated for one minute with the chopper and impeller on high speed; additional water and granulation time may be used to form proper granules. This composition was allowed to dry in a Niro Aeromatic Strea-1 Fluid Bed Dryer until the Loss on Drying (LOD) was less than one percent. The dried granulation was screened though a #16 Mesh sieve, then placed in an Aeromatic Fielder PP-1 High Shear Granulator equipped with a 10 L bowl. Meanwhile, Stearyl Alcohol was melted. While running the impeller at low speed, the melted Stearyl Alcohol was added; mixing was continued to achieve uniform distribution. Granulation continued at high speed until proper granules were formed, then the granules were cooled to room temperature. The cooled granules were screened through a #16 Mesh sieve and dispensed into a Patterson-Kelley P/K Blendmaster V-Blender. Stearic acid was added and the mixture was blended for five minutes. Talc was added and the mixture was blended for an additional five minutes. The blended granulation was compressed to 281.4 mg and 10 Kp hardness on a tablet press using a Stokes RB-2 5/16″ round standard concave beveled edge. The final tablet composition is listed below:

| Component | % | mg/tab |
|---|---|---|
| Albuterol Sulfate | 3.4 | 9.6 |
| Lactose | 71.1 | 200.0 |
| Stearyl Alcohol | 21.7 | 61.2 |
| Stearic Acid | 1.9 | 5.3 |
| Talc | 1.9 | 5.3 |
| Water* | 10-20 | 0.00 |

*Removed during processing

### Example 1

The ideal particle size for the uptake of a drug through the nasal mucosa is around 10 μm. Nasal aerosols are usually formulated to target a mean particle size of 10 μm, with a particle size distribution as narrow as possible. Particles below 10 μm would be expected to be exhaled out of the mouth. For maximum absorption of drugs into the lungs, an optimal mean particle size diameter of 2-5 μm is desirable.

As discussed above, the time available for absorption of drugs via the nasal route is limited due to the rapid clearance of the surface coating of the nasal mucosa. Therefore, the opioid in the opioid formulation of the invention is unlikely to reside for a period of time long enough to enable absorption into the nasal mucosa to take place. Tablet grinding of the opioid formulation of the invention will result in a powder having a wide range of particle sizes. However, some material around 10 μm, and a range between 10-250 μm, could be expected. It is unlikely that the ground powders would be optimized in the same way as proprietary formulations found in dry powder inhalers.

The experiments can be performed by substituting the Albuterol with other drugs (e.g., opioids, OxyContin®, or nifedipine). One skilled in the art will appreciate that the invention provides reduced potential for drug abuse due to the sustained release formulation of the invention, since it is the sustained release formulation that swells and forms a hydrophilic gel matrix upon exposure to liquids and it is the sustained release formulation that has muco-adhesive properties. Thus, a comparison of the sustained release formulation of the invention to conventional formulations (such as that used for OxyContin®) will provide the necessary comparison to demonstrate the unexpected results of the invention.

To demonstrate that the opioid formulations of the invention (e.g., an oxymorphone formulation) have an extremely poor deposition rate in the lungs when compared to commercially available opioid formulations (e.g., OxyContin®), the following experiment was conducted. Because the opioid formulations of the invention have an extremely poor deposition rate in the lungs when compared to commercially available opioid formulations, the opioid formulations of the invention will not provide the euphoria that commercially available opioid formulations provide, which means that the opioid formulations of the invention have significantly less potential for abuse when compared to conventional opioid formulations.

The use of a modified Twin Stage Impinger (BP Apparatus A) (hereafter “TSI”) for the evaluation of controlled release aerosol formulations (Drug Dev Ind Pharmacy, 26(11), 1191-1198 (2000), the disclosure of which is incorporated by reference herein in its entirety) has been previously shown to predict drug deposition and release from dry powder inhaler systems intended for pulmonary delivery. The TSI apparatus is sub-divided into two stages. The upper, or Stage 1 flask, captures particles greater than 6.8 μm using a conventional stage 1 jet diameter as specified in the British Pharmacopoeia. The Stage 2 flask adaptation captures all those particles less than 6.8 μm. In theory this could include some sub-micron material, though in practice such particles are usually drawn up through the pump exhaust.

Three tablets of Formulation 1 were ground for 5 minutes using a mortar and pestle, until a fine powder was obtained. Simple pestle and mortar grinding is unlikely to be able to facilitate the production of micronized powders. High pressure air jet milling would normally be required to do this. The sustained release delivery system of the invention is essentially ‘rubbery’ in nature, which means that the particles tend to bounce off each other rather than fracture on impact when a force is applied. Some small particles will result however, but the particle size range would be expected to be large, e.g., between 5-50 μm with a mean diameter of about 20 μm.

Approximately 50 mg of the ground Formulation 1 was weighed into a size 3 capsule. The capsule was inserted into the aerosol delivery device, a Rotohaler® (Glaxo Group Research Ltd.). The contents were discharged into the modified Stage 1 TSI, which was filled with approximately 263 mL of deionized water, so that the level of the water was just touching the screen. The contents of the Rotohaler® were then drawn through the TSI apparatus using a nominal pump flow rate of approximately 60 liters per minute. This rate is nominal based on previous calibration of the TSI, which was never intended as a model for either lung delivery of dry powder inhaler's or nasal delivery of the same. The Stage 1 flask was then removed and placed on a stirrer at 100 rpm to allow dissolution of the drug from the powder to commence. Samples in 5 mL aliquots were taken by syringe at 5 minutes, 10 minutes, 20 minutes, 25 minutes, 40 minutes, and 60 minutes. Fresh dissolution media (water) was replaced after each sampling point to enable the reservoir level to remain constant throughout the course of the experiment. A final sample was taken after the stirrer speed was set at maximum rpm to enable complete dissolution of all available drug to be facilitated. The experiment was repeated four times.

The dissolution experiment was repeated as described above for Formulation 2 and Formulation 3.

Drug release for all formulations was monitored by RP-HPLC using a Waters Spherisorb® C18 S5 ODS2 column (4.6×150 mm) (or equivalent) at 226 nm. The mobile phase comprised 90% of 1% glacial acetic acid, 9.5% methanol, 0.4% acetonitrile, and 0.1% triethylamine. The column temperature was set at 37° C. and the flow rate was 1.5 mL/min. To determine the percentage of drug released at each timepoint, the value of the same taken at that timepoint was compared to the value of the final sample that represented complete dissolution.

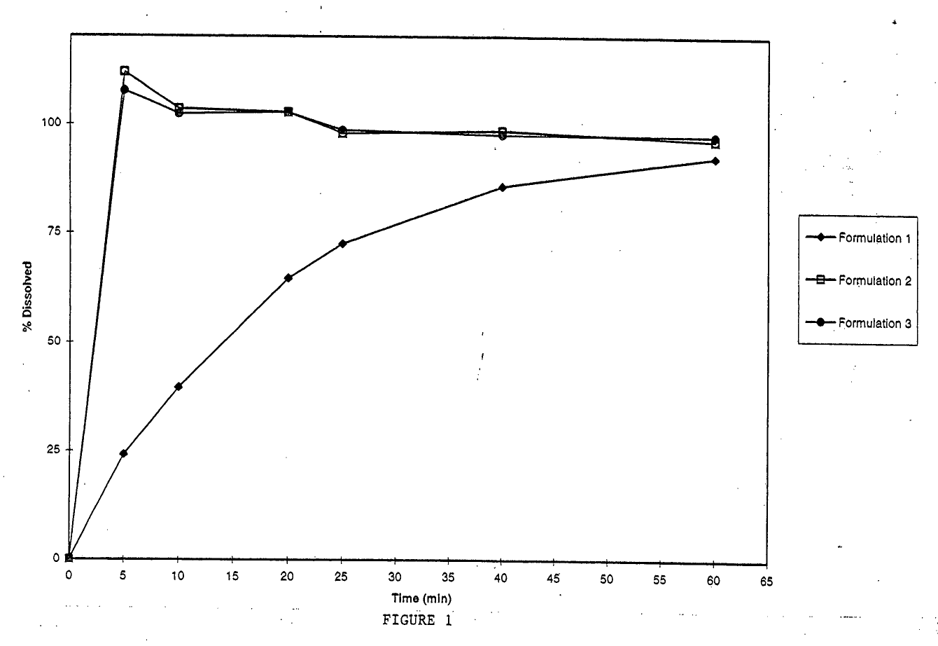

FIG. 1 is a graphical depiction of the dissolution profiles of Formulation 1, Formulation 2, Formulation 3. Formulation 2 and Formulation 3 depict complete (100%) dissolution within five minutes, leveling off for the remainder of the sixty-minute study. In comparison, Formulation 1 depicts a slower dissolution profile over the course of the sixty-minute study, with 92% of the material dissolved at 60 minutes.

All the Albuterol in Formulation 2 was released within the first five minutes. Similarly, all the Albuterol in Formulation 3 was released within the first five minutes. The Albuterol in Formulation 3 was released steadily over the course of one hour, with 92.4% dissolved at 60 minutes (Table 1).

TABLE 1

| | % Albuterol Dissolved (by HPLC) | | |
|---|---|---|---|
| Time | Formulation 1 | Formulation 2 | Formulation 3 |
| (min) | (SD) | (SD) | (SD) |
| 0 | 0.0 (0.0) | 0.0 (0.0) | 0.0 (0.0) |
| 5 | 24.2 (5.5) | 111.9 (1.9) | 107.7 (1.6) |
| 10 | 39.8 (6.8) | 103.5 (3.7) | 102.4 (2.2) |
| 20 | 64.7 (8.5) | 102.8 (3.9) | 102.8 (2.5) |
| 25 | 72.6 (7.4) | 97.9 (3.7) | 98.7 (2.0) |
| 40 | 85.7 (4.6) | 98.5 (2.0) | 97.6 (4.3) |
| 60 | 92.4 (1.7) | 96.3 (3.2) | 97.4 (3.0) |

### Example 2

To demonstrate that the opioid sustained release formulations of the invention (e.g., an oxymorphone formulation) have poor uptake into and discharge from a syringe when compared to commercially available opioid formulations (e.g., OxyContin®), the following experiment was conducted. Because the opioid formulations of the invention have an extremely poor uptake into and discharge from syringes when compared to commercially available opioid formulations, the opioid formulations of the invention do not provide easy access to the opioid and do not provide the euphoria that commercially available opioid formulations provide, which means that the opioid formulations of the invention have significantly less potential for abuse when compared to conventional opioid formulations.

The experiments can be performed by substituting the Albuterol with other drugs (e.g., opioids, OxyContin®, or nifedipine) that are more readily available. One skilled in the art will appreciate that the invention provides reduced potential for drug abuse due to the sustained release formulation of the invention, since it is the sustained release formulation that swells and forms a hydrophilic gel matrix upon exposure to liquids and it is the sustained release formulation that has muco-adhesive properties. Thus, a comparison of the sustained release formulation of the invention to conventional formulations (such as that used for OxyContin®) will provide the necessary comparison to demonstrate the unexpected results of the invention.

Seven tablets of Formulation 1 were crushed for 5 minutes using a mortar and pestle. The contents of the ground Formulation 1 were weighed, recorded, discharged into 140 ml of distilled water, and manually stirred to reduce clumping. The average weight of each tablet was 215.5 mg and the sample weight was 1.5085 g The solution was allowed to stand at room temperature for 5 minutes, stirring occasionally to prevent clumping.

Seven tablets of Formulation 2 were crushed for 5 minutes using a mortar and pestle. The contents of the ground Formulation 2 were weighed, recorded, discharged into 140 ml of distilled water, and manually stirred to reduce clumping. The average weight of each tablet was 286.8 mg and the sample weight was 2.0076 g. The solution was allowed to stand at room temperature for 5 minutes, stirring occasionally to prevent clumping.

Seven tablets of Formulation 3 were crushed for 5 minutes using a mortar and pestle. The contents of the ground Formulation 3 were weighed, recorded, discharged into 140 ml of distilled water, and manually stirred to reduce clumping. The average weight of each tablet was 284.1 mg and the sample weight was 1.9887 g The solution was allowed to stand at room temperature for 5 minutes, stirring occasionally to prevent clumping.

The viscosity of each formulation, prepared as described above, was measured using a Brookfield Model RVDV-III Rheometer rotational viscometer, equipped with a #RV4 spindle (or equivalent). Viscosity measurements were taken at 3 rpm, 6 rpm, 12 rpm, and 20 rpm.

The viscosity of Formulation 1 in water is significantly and unexpectedly higher than the viscosity of Formulation 2 or Formulation 3 (Table 2).

TABLE 2
Viscosity Measurement

| Sample | Spindle Speed | | Readings |
|---|---|---|---|
| Formulation 1 | 3 rpm | low | 1067.0 |
| | | high | 1267.0 |
| | | average | 1167.0 |
| | 6 rpm | low | 700.00 |
| | | high | 800.00 |
| | average | average | 750.00 |
| | 12 rpm | low | 483.00 |
| | | high | 500.00 |
| | | average | 491.50 |
| | 20 rpm | low | 350.00 |
| | | high | 360.00 |
| | | average | 355.00 |
| Formulation 2 | 3 rpm | low | 0.00 |
| | | high | 66.70 |
| | | average | 33.35 |
| | 6 rpm | low | 33.30 |
| | | high | 66.70 |
| | | average | 50.00 |
| | 12 rpm | low | 0.00 |
| | | high | 33.30 |
| | | average | 16.65 |
| | 20 rpm | low | 0.00 |
| | | high | 10.00 |
| | | average | 5.00 |
| Formulation 3 | 3 rpm | low | 0.00 |
| | | high | 66.70 |
| | | average | 33.35 |
| | 6 rpm | low | 33.30 |
| | | high | 66.70 |
| | | average | 50.00 |
| | 12 rpm | low | 0.00 |
| | | high | 16.70 |
| | | average | 8.35 |
| | 20 rpm | low | 0.00 |
| | | high | 0.00 |
| | | average | 0.00 |

The patents, patent applications, and publications cited herein are incorporated by reference herein in their entirety.

Various modifications of the invention, in addition to those described herein, will be apparent to one skilled in the art from the foregoing description. Such modifications are intended to fall within the scope of the appended claims.

What is claimed is:

1. A method for relieving pain comprising administering to a patient in need thereof a reduced abuse potential solid dosage formulation comprising an opioid mixed with a granulated sustained release delivery system,
wherein the granulated sustained release delivery system comprises at least one heteropolysaccharide gum, at least one homopolysaccharide gum, and at least one pharmaceutical diluent.

2. The method of claim 1, wherein the reduced abuse potential solid dosage formulation forms a gel matrix with muco-adhesive properties when crushed or powdered upon contact with a fluid.

3. The method of claim 1, wherein the reduced abuse potential solid dosage formulation forms a viscous solution when crushed or powdered upon contact with a fluid.

4. The method of claim 1, wherein the granulated sustained release delivery system further comprises at least one hydrophobic polymer.

5. The method of claim 1, wherein the granulated sustained release delivery system further comprises at least one cationic cross-linking compound selected from monovalent cations, multivalent cations, and salts.

6. The method of claim 5, wherein the cationic cross-linking agent is selected from the group consisting of: alkali metal sulfate, alkali metal chloride, alkali metal borate, alkali metal bromide, alkali metal citrate, alkali metal acetate, alkali metal lactate, alkaline earth metal sulfate, alkaline earth metal chloride, alkaline earth metal borate, alkaline earth metal bromide, alkaline earth metal citrate, alkaline earth metal acetate, alkaline earth metal lactate and a mixture thereof.

7. The method of claim 5, wherein the cationic cross-linking agent is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride and a mixture thereof.

8. The method of claim 1, wherein the reduced abuse potential solid dosage formulation further comprises an outer coating, wherein the outer coating comprises at least one hydrophobic polymer.

9. The method of claim 1, wherein the reduced abuse potential solid dosage formulation further comprises an outer coating, wherein the outer coating comprises at least one plasticizer.

10. The method of claim 1, wherein the opioid is a mu-agonist or a mixed mu-agonist/antagonist.

11. The method of claim 1, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

12. The method of claim 1, wherein the solid dosage formulation is a tablet.

13. A method for relieving pain comprising administering to a patient in need thereof a reduced abuse potential solid dosage formulation comprising an opioid mixed with a granulated sustained release delivery system,
wherein the granulated sustained release delivery system comprises at least one heteropolysaccharide gum, at least one homopolysaccharide gum, at least one pharmaceutical diluent, and at least one cationic cross-linking agent selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride and a mixture thereof.

14. The method of claim 13, wherein the at least one heteropolysaccharide gum is xanthan gum.

15. The method of claim 14, wherein the at least one homopolysaccharide gum is locust bean gum or guar gum.

16. The method of claim 15, wherein the at least one cationic cross-linking agent is calcium sulfate.

17. The method of claim 16, wherein the solid dosage formulation is a tablet.

18. A method for relieving pain comprising administering to a patient in need thereof a reduced abuse potential solid dosage formulation comprising oxymorphone mixed with a granulated sustained release delivery system,
wherein the granulated sustained release delivery system comprises at least one heteropolysaccharide gum, at least one homopolysaccharide gum, and at least one pharmaceutical diluent.

19. The method of claim 18, wherein the reduced abuse potential solid dosage formulation forms a gel matrix with muco-adhesive properties when crushed or powdered upon contact with a fluid.

20. The method of claim 18, wherein the reduced abuse potential solid dosage formulation forms a viscous solution when crushed or powdered upon contact with a fluid.

21. The method of claim 18, wherein the granulated sustained release delivery system further comprises at least one hydrophobic polymer.

22. The method of claim 18, wherein the granulated sustained release delivery system further comprises at least one cationic cross-linking compound selected from monovalent cations, multivalent cations, and salts.

23. The method of claim 22, wherein the cationic cross-linking agent is selected from the group consisting of: alkali metal sulfate, alkali metal chloride, alkali metal borate, alkali metal bromide, alkali metal citrate, alkali metal acetate, alkali metal lactate, alkaline earth metal sulfate, alkaline earth metal chloride, alkaline earth metal borate, alkaline earth metal bromide, alkaline earth metal citrate, alkaline earth metal acetate, alkaline earth metal lactate and a mixture thereof.

24. The method of claim 22, wherein the cationic cross-linking agent is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride and a mixture thereof.

25. The method of claim 18, wherein the reduced abuse potential solid dosage formulation further comprises an outer coating, wherein the outer coating comprises at least one hydrophobic polymer.

26. The method of claim 18, wherein the reduced abuse potential solid dosage formulation further comprises an outer coating, wherein the outer coating comprises at least one plasticizer.

27. The method of claim 18, wherein the solid dosage formulation is a tablet.

28. A method for relieving pain comprising administering to a patient in need thereof a reduced abuse potential solid dosage formulation comprising oxymorphone mixed with a granulated sustained release delivery system,
wherein the granulated sustained release delivery system comprises at least one heteropolysaccharide gum, at least one homopolysaccharide gum, at least one pharmaceutical diluent, and at least one cationic cross-linking agent selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride and a mixture thereof.

29. The method of claim 28, wherein the at least one heteropolysaccharide gum is xanthan gum.

30. The method of claim 29, wherein the at least one homopolysaccharide gum is locust bean gum or guar gum.

31. The method of claim 30, wherein the at least one cationic cross-linking agent is calcium sulfate.

32. The method of claim 31, wherein the solid dosage formulation is a tablet.
